(19) United States
(12) Patent Application Publication
(10) Pub. No.: US 2011/0313040 A1
(43) Pub. Date: Dec. 22, 2011

(54) ORGANIC NUTRIENT SALTS, METHODS OF PREPARATION AND USES

(75) Inventors: Xianqi Kong, Dollard-des-Omeaux (CA); Nigel Levens, Beaconsfield (CA); Serge Lamothe, Boisbriand (CA); Ahmed Aman, Oakville (CA)

(73) Assignee: BELLUS HEALTH INC., Laval (CA)

(21) Appl. No.: 13/129,243

(22) PCT Filed: Nov. 13, 2009
(86) PCT No.: PCT/CA09/01653

§ 371 Date: Sep. 2, 2011

Related U.S. Application Data
(60) Provisional application No. 61/114,822, filed on Nov. 14, 2008.

(51) Int. Cl. A61K 31/205; C07C 303/44; A23L 1/304; A61P 25/00; A61P 25/28; A23L 1/237; C07C 309/14; C07C 303/02

(52) U.S. Cl. 514/554; 562/104; 426/649; 426/2

(57) ABSTRACT

The invention relates to organic nutrient salts, to compositions containing the same, to their preparation as well as their use. Preferred compositions include nutraceutical compositions and dietary supplements for use in neuroprotection and for improving or protecting cognitive and memory function.

### RELATED APPLICATION

This application claims priority under 35 USC 119(e) from U.S. provisional patent application No. 61/114,822 filed Nov. 14, 2008 which are incorporated herein by reference.

### FIELD OF THE INVENTION

The invention relates to organic nutrient salts comprised of 3-amino-1-propanesulfonic acid (homotaurine) and a cationic component which contributes to the therapeutic and/or nutritional value of the organic nutrient salt, their methods of production and their use as nutraceuticals. The invention encompasses compositions comprising the organic nutrient salts, for example, nutraceutical compositions, or nutritional or dietary supplements. The composition is also useful as a nutraceutical for maintaining health, for neuroprotection, for improving or preserving cognitive, memory and brain function, or for preventing or treating a disease or condition involving beta-amyloid deposition or neuronal cell toxicity.

### BACKGROUND OF THE INVENTION

Homotaurine occurs naturally in various edible seaweeds and is known to be useful for protecting the brain structure associated with memory and learning, protecting memory function, sustaining brain cell health, maintaining verbal skills and comprehension ability, as well as supporting planning and execution skills.

Inorganic salts of homotaurine have been described, for example sodium (see WO1996/028187, incorporated by reference in its entirety) and strontium (see WO 2007/116149, incorporated by reference in its entirety). However, the sodium salt of homotaurine when tested in humans caused stomach and gastrointestinal intolerance (e.g. nausea, vomiting).

Homotaurine is usually consumed in seaweed, which has the benefit of providing additional nutrients such as amino acids, fibers, etc. However, the concentration of homotaurine in seaweeds is low and variable making it difficult to control the amounts of homotaurine ingested to obtain the optimal health benefit from homotaurine. Also, seaweeds although generally consumed in some parts of the globe, are not necessarily part of everyone's diet.

It is desirable to obtain a composition with improved ease of administration, providing additional nutrients, and/or lessening gastrointestinal side effects without increasing sodium intake.

### SUMMARY OF THE INVENTION

The invention includes organic nutrient salts, methods for their preparation and their use as nutraceuticals. These organic nutrient salts have the general formula:

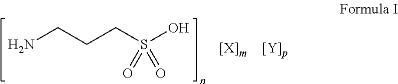

wherein

X is an organic component comprising at least one basic, acidic, cationic or anionic moiety;

Y is an additional component selected from the group consisting of organic or inorganic salt-forming ions, or absent;

m and n are each independently an integer selected from the group consisting of 1, 2, and 3; and

p is an integer selected from the group consisting of 0, 1, 2, and 3, wherein when p is 0, then Y is absent;

or a nutraceutically acceptable salt thereof.

In one aspect, the invention relates to organic nutrient salts of Formula I, wherein X is (a) an organic component comprising a basic moiety; (b) an organic component comprising cationic moiety; (c) an organic component comprising both a basic and an acidic moiety; or (d) an organic component comprising both a cationic and an acidic moiety. The invention further relates to organic nutrient salts of Formula I, wherein X is an organic component comprising a basic and/or cationic moiety, m and n are both 1, and p is 0, 1 or 2, preferably 0 or 1. The invention further relates to organic nutrient salts of Formula I, wherein Y comprises (a) an alkaline or alkaline-earth ion, e.g. magnesium, calcium, sodium, potassium, and the like; (b) an ammonium ion; (c) a halide ion, e.g. chloride, bromide, and iodide or (d) an organic acid ion, e.g. acetate. In one aspect of the invention, p is 0.

In one aspect of the invention, the organic nutrient salt has the formula:

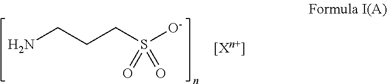

### Wherein,

X is an organic component comprising a cationic or protonated basic moiety; and

n is an integer selected from the group consisting of 1, 2, and 3; or a nutraceutically acceptable salt thereof.

The present invention relates to an organic nutrient salt of Formula I or I(A), wherein n is 1 or 2, preferably n is 1. The invention also relates to the organic nutrient salt of Formula I or I(A), wherein X is an organic base, preferably a strong organic base. In one aspect, the invention relates to organic nutrient salts where X is a nutraceutically acceptable organic base. In another aspect, the invention relates to organic nutrient salts of Formula I or I(A), wherein X is a naturally occurring component compatible with human consumption, preferably X has beneficial health properties. The invention also relates to organic nutrient salts where X further provides desired nutraceutical properties to the salt composition.

The invention further relates to the organic nutrient salt of Formula I or I(A), wherein X is a quaternary ammonium compound, e.g. L-carnitine and L-carnitine alkanoyl derivatives (e.g. acetyl, propionyl and butyryl-L-carnitine), choline and choline derivatives (e.g. acetylcholine, butyrylcholine, propionylcholine, and other aliphatic esters of choline, fatty acid esters of choline (such as eicosapentaenoyl (EPA), docosahexaenoyl (DHA), docosapentaenoyl (DPA), capryloyl, lauroyl, myristoyl, palmytoyl, stearoyl, oleoyl, ricinoleoyl, linoleoyl, alpha-linolenoyl, and arachidonoyl), phosphorylcholine, and phosphatidylcholines). The invention also relates to the organic nutrient salt of Formula I or I(A), wherein X is a betaine, e.g. L-carnitine, L-carnitine alkanoyl derivatives (e.g. acetyl, propionyl and butyryl-L-carnitine), and trimethylglycine. The invention further relates to the organic nutrient salt of Formula I or I(A), wherein X is an amino acid, e.g. L-arginine, L-lysine, histidine, and the like. The invention further relates to the organic nutrient salt of Formula I or I(A), wherein X is amine-containing or polyamine compound, e.g putrescine, spermidine, spermine, galanthamine, dimethylaminoethanol, and the like. The invention also further relates to the organic nutrient salt of Formula I or I(A), wherein X is a vitamin, e.g. vitamins B1 (thiamine), B2 (riboflavin), B3 (niacin or nicotinic acid), B4 (adenine), B6 (pyridoxine), B12 (cobalamine), vitamin U (S-methylmethionine) or folic acid. The invention also further relates to the organic nutrient salt of Formula I or I(A), wherein X is an alkaloid, e.g. huperzine A and tetrandrine. In one embodiment, X is selected from the group consisting of choline or a choline derivative, L-carnitine or a L-carnitine derivative, and L-Arginine. In yet another embodiment, X is selected from the group consisting of nicotinic acid, gamma-aminobutyric acid, L-histidine, L-lysine, glucosamine, L-proline, huperzine A, tetrandrine, and guanine.

The invention further relates to an organic nutrient salt selected from the group consisting of carnitine homotaurinate, acetylcarnitine homotaurinate, propionylcarnitine homotaurinate and butyryl-L-carnitine homotaurinate, choline homotaurinate, acetylcholine homotaurinate, butyrylcholine homotaurinate, phosphorylcholine homotaurinate, L-arginine homotaurinate, L-lysine homotaurinate, histidine homotaurinate, trimethylglycine homotaurinate, putrescine homotaurinate, spermidine homotaurinate, spermine homotaurinate, galanthamine homotaurinate, dimethylaminoethanol homotaurinate, thiamine homotaurinate, riboflavin homotaurinate, nicotinic acid homotaurinate, adenine homotaurinate, pyridoxine homotaurinate, cobalamine homotaurinate, S-methylmethionine homotaurinate, folic acid homotaurinate, huperzine A homotaurinate, tetrandrine homotaurinate, and acceptable salts, solvates and polymorphs thereof. In one aspect, the invention relates to choline homotaurinate and its nutraceutically acceptable salts, for such as choline homotaurinate hydrochloride, choline homotaurinate acetic acid salt, and the like.

The present invention also relates to the process for forming an organic nutrient salt, said process comprising the step of (a) dissolving homotaurine and at least one organic, salt-forming, component in a solvent together or successively to obtain a solution, and (b) concentrating the solution. Another aspect of the invention also relates to the process for forming an organic nutrient salt, said process comprising the step of (a) dissolving homotaurine or a salt thereof and a second organic, salt-forming, component in a solvent together or successively to obtain a solution, (b) concentrating and/or cooling the solution until crystals or powder appear, and (c) filtering and drying the crystals or powder obtained. A further aspect of the invention also relates to the process for forming an organic nutrient salt, said process comprising the step of (a) dissolving homotaurine or a salt thereof and a second organic, salt-forming, component in a solvent together or successively to obtain a solution, (b) adding a second solvent to the solution until crystals appear or a powder precipitates, and (c) filtering and drying the crystals or powder obtained.

The invention also relates to the process of producing choline homotaurinate, and its nutraceutically acceptable salts, the process comprising the steps of: (a) choline and its counterion and homotaurine (including its zwitterionic form) or a salt form thereof are mixed together in a solution; and (b) is concentrated in vacuo and a solid is formed. In one aspect, the process further comprises step (c) purifying the solid. In one aspect, the purifying step comprises recrystallizing the solid from water, an organic solvent or a miscible mixture thereof, preferably a lower alkyl alcohol solvent, more preferably ethanol or isopropanol, most preferably isopropanol. In one aspect, the choline counteranion is selected from chloride, hydroxide, acetate, bicarbonate, tartrate, and the like, preferably hydroxide.

Any of the organic nutrient salts of the invention may be used as is or may be formulated in a nutraceutical or as a dietary supplement. The preparation may be in a suitable form for direct administration, such as caplets, pills, and the like, or in a powder form to be added to food, drinks, and the like. Accordingly, the present invention equally encompasses the organic nutrient salts, nutraceutical and dietary or nutritional supplements compositions containing them, as well as methods for employing them in neuroprotection, improving or preserving cognitive and memory function.

In one aspect, the organic nutrient salts and compositions of the invention are useful for protecting memory function. In another aspect, the organic nutrient salts and compositions of the invention are useful for protecting the brain structure associated with memory and learning, for preserving memory, for sustaining brain cell health, for maintaining verbal skills and comprehension ability and to support planning and execution skills.

The invention also relates to a method for providing neuroprotection to a subject comprising administering to the subject a nutraceutically effective amount of an organic nutrient salt or composition of the invention, such that neuroprotection is provided to the subject. The invention further relates to a composition of the invention for use in the treatment or prevention of inflammation, neuronal cell toxicity, neuronal cell death or neuronal cell loss in a subject having a condition or disease in which Aβ amyloidogenic proteins and peptides are present, or being susceptible or predisposed to said condition or disease, preferably, the disease or condition is characterized by Aβ deposition, as well as in the treatment or prevention of diseases and conditions such as Alzheimer's disease, cerebral amyloid angiopathy, Down's syndrome, mild cognitive impairment, mild-to-moderate cognitive impairment, aging of the brain, age-associated cognitive impairment, age-associated memory impairment.

The invention also relates to a method for reducing side effects of homotaurine in a human subject (e.g., reducing or preventing gastrointestinal intolerance), wherein homotaurine is administered as a component of an organic nutrient salt, which yields or generates homotaurine after being administered to said human subject.

Additional objects, advantages and features of the present invention will become more apparent upon reading of the following non-restrictive description of preferred embodiments which are exemplary and should not be interpreted as limiting the scope of the invention.

### DESCRIPTION OF THE FIGURE

FIG. 1: shows the ¹H NRM spectrum of the organic nutrient salt of carnitine and homotaurine obtained from the procedure of Example 1. NRM spectrum was performed in D₂O on an Inova-500 apparatus at 500 MHz.

### DETAILED DESCRIPTION OF THE INVENTION

### I. Definitions

All technical and scientific terms used herein have the same meaning as commonly understood by one ordinary skilled in the art to which the invention pertains. For convenience, the meaning of certain terms and phrases used herein are provided below.

To the extent the definitions of terms in the publications, patents, and patent applications incorporated herein by reference are contrary to the definitions set forth in this specification, the definitions in this specification control. The section headings used herein are for organizational purposes only, and are not to be construed as limiting the subject matter disclosed.

It should be noted that, the singular forms “a”, “an”, and “the” include plural referents unless the content clearly dictates otherwise. Thus, for example, reference to a composition containing “a compound” or “a salt” includes a mixture of two or more compounds and salts respectively. It should also be noted that the term “or” is generally employed in its sense including “and/or” unless the content clearly dictates otherwise.

The chemical structures herein are drawn according to the conventional standards known in the art. Thus, where an atom, such as a carbon atom, as drawn appears to have an unsatisfied valency, then that valency is assumed to be satisfied by a hydrogen atom even though that hydrogen atom is not necessarily explicitly drawn. Hydrogen atoms should be inferred to be part of the compound.

As used herein, the term “homotaurine”, “first component” and equivalent expressions refers to 3-amino-1-propanesulfonic acid, its zwitterionic form, and acceptable salts and solvates thereof. The compound may be of natural source (extracted or purified from a natural source, e.g. a seaweed) or may be synthetic (prepared or provided by a commercial source). The term further includes natural extracts containing at least 10%, at least 20%, at least 30%, at least 40%, at least 50%, at least 60%, at least 70%, at least 80%, at least 85%, at least 90%, at least 95%, at least 98%, or at least 99% of homotaurine in the dried extract. In general, the compound may be hydrated or solvated. The compound may exist in multiple crystalline or amorphous forms. In general, all physical forms are equivalent for the uses contemplated herein and are intended to be within the scope of the present invention.

As used herein, the terms “second component”, or “additional component” and equivalent expressions refer to a molecule which will form a salt when in contact with homotaurine. The terms equally refer to molecules having a basic moiety capable of forming a salt with homotaurine, or a molecule having an intrinsic positive charge (cation). As used herein, the term “betaine” refers to a neutral compound with a positively charged cationic functional group such as ammonium ion which bears no hydrogen atom and with a negatively charged functional group such as a carboxylate group. Examples of betaine include, without limitation, carnitine, alkanoyl derivatives of carnitine and trimethylglycine.

As used herein, the term “component” generally refers to any or all of the molecules forming the salt.

As used herein, the terms “organic salt”, “nutrient salt” and “nutrient organic salt”, and equivalent expressions refer to a salt represented by Formula I or I(A), comprising 3-amino-1-propanesulfonic acid and at least one additional organic component having a cationic charge or basic moiety, together ionically associated. The term also includes zwitterionic forms of the salt, for example, when carnitine homotaurinate has a positive charge on the respective nitrogen atoms of each component (i.e, —NH₃⁺ and —N(CH₃)₃⁺), and a negative charge on each of their acidic counterparts (i.e, —SO₃⁻ and —CO₂⁻). The term also equally includes nutraceutically acceptable salts of the salts, for example choline homotaurinate hydrochloride, which is the hydrochloride salt of the salt formed by choline and homotaurine. The term also further includes any solvated or hydrated form of the salts formed as well as their crystalline and amorphous forms. In general, all physical forms are equivalent for the uses contemplated herein and are intended to be within the scope of the present invention.

The term “organic component”, “organic compound” or “organic molecule” and equivalent expressions refer to a component, compound or molecule comprising a component having at least one carbon atom in its structure, and excluding carbonates (e.g. sodium bicarbonate), carbon oxides (e.g. carbon dioxide), cyanides (e.g. potassium cyanide), as well as the allotropes of carbon (e.g. carbon and graphite).

The term “organic” when qualifying a salt or nutrient salt, refers to the salt as having at least one carbon atom in the structure of at least two of its components, and excluding carbonates (e.g. sodium bicarbonate), carbon oxides (e.g. carbon dioxide), cyanides (e.g. potassium cyanide), as well as the allotropes of carbon (e.g. carbon and graphite).

A “nutraceutically acceptable salt”, “acceptable salt” or “suitable salt” of a component or organic nutrient salt means a salt of a component or organic nutrient salt that is acceptable for human consumption. Desirable are salts that retain or improve the biological and/or chemical and/or physical properties of the free acids and bases of the parent component as defined herein or that takes advantage of an intrinsically basic, acidic or charged functionality on the molecule and that is not biologically or otherwise undesirable. Example of acceptable salts are also described, for example, in Berge et al., J. Pharm. Sci. 66, 1-19 (1977). Such salts include:

(1) acid addition salts are formed on an amine group or equivalent by the addition of inorganic acids such as hydrochloric acid, hydrobromic acid, hydroiodic acid, sulfuric acid, sulfamic acid, nitric acid, phosphoric acid, carbonate forming agents, and the like; or formed with organic acids such as acetic acid, propionic acid, lactic acid, oxalic, glycolic acid, pivalic acid, t-butylacetic acid, β-hydroxybutyric acid, valeric acid, hexanoic acid, cyclopentanepropionic acid, pyruvic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, cyclohexylaminosulfonic acid, benzenesulfonic acid, sulfanilic acid, 4-chlorobenzenesulfonic acid, 2-napthalenesulfonic acid, 4-toluenesulfonic acid, camphorsulfonic acid, 3-phenyl propionic acid, lauryl sulphonic acid, lauryl sulfuric acid, oleic acid, palmitic acid, stearic acid, lauric acid, embonic (pamoic) acid, palmoic acid, pantothenic acid, lactobionic acid, alginic acid, galactaric acid, galacturonic acid, gluconic acid, glucoheptonic acid, glutamic acid, naphthoic acid, hydroxynapthoic acid, salicylic acid, ascorbic acid, stearic acid, muconic acid, and the like;

(2) base addition salts are formed when the acidic proton of a sulfonic acid, phosphonic acid, carboxylic acid and the like, of the parent components is replaced by a metal ion, including, an alkali metal ion (e.g. lithium, sodium, potassium), an alkaline earth ion (e.g. magnesium, calcium, barium), or other metal ions such as aluminum, zinc, iron and the like; or coordinates with an organic base such as ammonia, ethylamine, diethylamine, ethylenediamine, N,N′-dibenzylethylenediamine, ethanolamine, diethanolamine, triethanolamine, tromethamine, N-methylglucamine, piperazine, chloroprocain, procain, choline, lysine and the like.

Acceptable salts may be prepared from the parent agent by conventional chemical methods. Generally, such salts are prepared by reacting the free acid or base forms of these agents with a stoichiometric amount of the appropriate base or acid in water or in an organic solvent, or in a mixture of the two. Salts may be prepared in situ, during the final isolation or purification of the agent or by separately reacting a purified compound of the invention in its free acid or base form with the desired corresponding base or acid, and isolating the salt thus formed. The terms also include zwitterionic compounds containing a cationic group covalently bonded to an anionic group, as they are “internal salts” or “inner salts”.

All acid, salt, base, and other ionic and non-ionic forms of the compounds and components described are included in the compositions, preparations and methods and uses of the invention. For example, if the compound or component is shown as an acid herein, the salt forms of the compound or component are also included. Likewise, if the compound or component is shown as a salt, the acid and/or basic forms are also included.

“Abeta”, “Aβ”, or “β-amyloid”, is defined as any peptide resulting from beta-secretase mediated cleavage of Beta Amyloid Precursor Protein (APP), including for examples peptides of 37, 38, 39, 40, 41, 42, and 43 amino acids, and extending from the beta-secretase cleavage site to amino acids 37, 38, 39, 40, 41, 42, or 43. It also includes It also includes N-terminal truncated species of above peptides, such as the pyroglutamic forms pE3-40, pE3-42, pE3-43, pE11-42, pE11-43 and the like. For convenience of nomenclature, “Aβ₁₋₄₂”, may be referred to herein as “Aβ(1-42)” or simply as “Aβ₄₂” (and likewise for any other amyloid peptides discussed herein). As used herein, the terms “Abeta”, “Aβ”, “β-amyloid”, “amyloid-β” are synonymous referring collectively to truncated and non-truncated peptide species of the sequence between β- and γ-cleavage sites of APP.

The term “amyloid-β disease or condition” or “amyloid-β related disease or condition” may be used for mild cognitive impairment; vascular dementia; early Alzheimer's disease; Alzheimer's disease, including sporadic (non-hereditary) Alzheimer's disease and familial (hereditary) Alzheimer's disease; cerebral amyloid angiopathy (“CAA”); hereditary cerebral hemorrhage; senile dementia; Down's syndrome; inclusion body myositis (“IBM”); age-related macular degeneration (“ARMD”); mild-to-moderate cognitive impairment or mild cognitive impairment (“MCI”); and conditions associated with aging, such as age associated memory impairment (AAMI).

As used herein the term “effective amount” refers to the amount of the organic nutrient salt, its content in homotaurine or in the additional nutrient, or a composition thereof, upon single or multiple administration to or consumption by the subject, which provides the desired effect to the subject. An effective amount can be readily determined by the use of known techniques and/or by observing results obtained under analogous circumstances. In determining the effective amount or dose administered, a number of factors are considered, including, but not limited to: the size, age, and general health of the subject; the specific disease involved; the degree of or involvement or the severity of the disease; the response of the individual subject; the mode of administration; the bioavailability characteristics of the preparation administered; the use of concomitant medication; and other relevant circumstances. The effective amount refers to an amount of the organic nutrient salt, its content in homotaurine or in the additional nutrient, or composition to obtain significant benefit to the subject by providing neuroprotection, protecting memory function, protecting the brain structure associated with memory and learning, by preserving memory, by sustaining brain cell health, by maintaining verbal skills and comprehension ability and/or by supporting planning and execution skills.

More generally, the terms lessening etc., increasing etc., refer in context herein to the percentage changes, e.g., by 5%, 10%, 20%, 30%, 40%, 50%, 60%, 70%, 80%, 90%, 95%, 99%, 125%, etc., or even more, e.g., 2, or 4 fold, or even more.

“Nutraceutically acceptable” and equivalent expressions refer to salts, inert ingredients, excipients, additives, which the term describes, suitable for use in contact with the tissues of humans and animals without undue toxicity, incompatibility, instability, irritation, and the like, commensurate with a reasonable benefit/risk ratio. In general, the term “nutraceutically acceptable” includes what is commonly used and generally accepted as safe by the nutraceutical industry. It preferably refers to a compound or composition that is approved or approvable by a regulatory agency of the Federal or state government or listed in the U.S. Pharmacopoeia or other generally recognized pharmacopoeia or Food administration or have been generally used as a dietary ingredient in animals and more particularly in humans.

“Acceptable vehicle” or “nutraceutically acceptable vehicle” refer to a diluent, adjuvant, excipient, or carrier with which an organic nutrient salt of the invention is administered.

“Composition” or “nutraceutical composition” refer to the organic nutrient salt of the invention in combination with at least one nutraceutically acceptable vehicle, with which the organic nutrient salt is administered to a subject.

The terms “supplement”, “nutraceutical supplement”, “dietary supplement”, “food supplement” and equivalent expressions both refer to the organic nutrient salt itself and to a composition as defined above either in a dosage form (e.g. capsules, pills, caplets, etc) or a food additive (e.g. power) to be added to edible material prior to consumption.

“Preventing” or “prevention” is intended to refer at least the reduction of likelihood of the risk of (or susceptibility to) acquiring a disease, disorder or condition (i.e., causing at least one of the clinical symptoms of the disease, disorder or condition not to develop in a patient that may be exposed to or predisposed to the disease but does not yet experience or display symptoms of the disease).

“Treating” or “treatment” of any disease, disorder or condition refers, in some embodiments, to amelioration of at least one disease, disorder or condition (i.e., arresting or reducing the development of the disease, disorder or condition or at least one of the clinical symptoms thereof). In certain embodiments “treating” or “treatment” refers to ameliorating at least one physical parameter, which may or may not be discernible by the patient. In certain embodiments, “treating” or “treatment” refers to inhibiting the disease, disorder or condition, either physically, (e.g., stabilization of a discernible symptom), physiologically (e.g., stabilization of a physical parameter), or both. In certain embodiments, “treating” or “treatment” refers to delaying the onset of the condition, disease or disorder. The term “treating” refers to any indicia of success in the treatment or amelioration of an injury, pathology or condition, including any objective or subjective parameter such as abatement; remission; diminishing of symptoms or making the injury, pathology or condition more tolerable to the subject; slowing in the rate of degeneration or decline; making the final point of degeneration less debilitating; improving a subject's physical or mental well-being; improving or preserving memory and/or cognitive functions; restoring and/or improving alertness and ability to concentrate or, in some situations, preventing the onset of dementia. The treatment or amelioration of symptoms can be based on objective or subjective parameters; including the results of a physical examination, a psychiatric evaluation, or a cognition test such as CDR, MMSE, DAD, ADAS-Cog, or a subscale thereof (e.g. subset of tests related to memory), or another test known in the art.

Reference will now be made in detail to certain embodiments of organic nutrient salts, components and methods. The disclosed embodiments are not intended to be limiting of the invention.

### II. Organic Nutrient Salts

The present invention provides an organic nutrient salt comprising homotaurine as a first component and at least one second organic component comprising a cationic moiety (intrinsic cation or protonated base), methods of producing them as well as their use as nutraceuticals. The organic nutrient salt of the invention is a salt of the Formula:

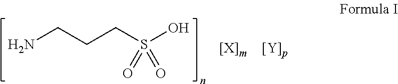

wherein,

X is an organic component comprising at least one basic, acidic, cationic or anionic moiety;

Y is an additional component selected from the group consisting of organic or inorganic salt-forming ions, or absent;

m and n are each independently an integer selected from the group consisting of 1, 2, and 3; and

p is an integer selected from the group consisting of 0, 1, 2, and 3, wherein when p is 0, then Y is absent;

or a nutraceutically acceptable salt or solvate thereof.

In one aspect, the invention relates to organic nutrient salts of Formula I, wherein X is (a) an organic component comprising a basic moiety; (b) an organic component comprising cationic moiety; (c) an organic component comprising both a basic and an acidic moiety; or (d) an organic component comprising both a cationic and an acidic moiety. The invention further relates to organic nutrient salts of Formula I, wherein X is an organic component comprising a basic and/or cationic moiety, m and n are both 1, and p is 0, 1 or 2, preferably 0 or 1. The invention further relates to organic nutrient salts of Formula I, wherein Y comprises (a) an alkaline or alkaline-earth ion, e.g. magnesium, calcium, sodium, potassium, and the like; (b) an ammonium ion; (c) a halide ion, e.g. chloride, bromide, and iodide or (d) an organic acid ion, e.g. acetate. In one aspect of the invention, p is 0 and Y is absent.

In one aspect of the invention, the organic nutrient salt has the formula:

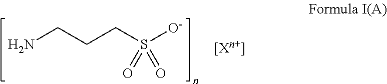

### Wherein,

X is an organic component comprising a cationic or protonated basic moiety; and

n is an integer selected from the group consisting of 1, 2, and 3;

or a nutraceutically acceptable salt or solvate thereof.

The present invention relates to an organic nutrient salt of Formula I or I(A), wherein n is 1 or 2, preferably n is 1. The invention also relates to the organic nutrient salt of Formula I or I(A), wherein X is an organic base, preferably a strong organic base. In one aspect, the invention relates to organic nutrient salts of Formula I or I(A), wherein X is a nutraceutically acceptable organic base. In another aspect, the invention relates to organic nutrient salts of Formula I or I(A), wherein X is a naturally occurring component compatible with human consumption, preferably X has known beneficial health properties. The invention also relates to organic nutrient salts where X further provides desired nutraceutical properties to the salt composition.

The invention further relates to the organic nutrient salt of Formula I or I(A), wherein X is a quaternary ammonium compound, e.g. L-carnitine and L-carnitine alkanoyl derivatives (e.g. acetyl, propionyl and butyryl-L-carnitine), choline and choline derivatives (e.g. acetylcholine, butyrylcholine and phosphorylcholine). The invention also relates to the organic nutrient salt of Formula I or I(A), wherein X is a betaine, e.g. L-carnitine, L-carnitine alkanoyl derivatives (e.g. acetyl, propionyl and butyryl-L-carnitine), and trimethylglycine. The invention further relates to the organic nutrient salt of Formula I or I(A), wherein X is an amino acid, e.g. L-arginine, L-lysine, histidine, and the like. The invention further relates to the organic nutrient salt of Formula I or I(A), wherein X is amine-containing or polyamine compound, e.g. putrescine, spermidine, spermine, galanthamine, dimethylaminoethanol and the like. The invention also further relates to the organic nutrient salt of Formula I or I(A), wherein X is a vitamin, e.g. vitamins B1 (thiamine), B2 (riboflavin), B3 (niacin or nicotinic acid), B4 (adenine), B6 (pyridoxine), vitamin U (S-methylmethionine) or folic acid. The invention also further relates to the organic nutrient salt of Formula I or I(A), wherein X is an alkaloid, e.g. huperzine A and tetrandrine. In one embodiment, X is selected from the group consisting of choline or a choline derivative, L-carnitine or a L-carnitine derivative, and L-Arginine. In yet another embodiment, X is selected from the group consisting of nicotinic acid, gamma-aminobutyric acid, L-histidine, L-lysine, glucosamine, L-proline, huperzine A, tetrandrine, and guanine.

### III. Methods of Preparation of the Organic Nutrient Salts

The invention relates to processes for the preparation of the organic nutrient salts of the invention. The organic nutrient salts are prepared by any procedure generally known to the skilled in the art. Generally, a salt is formed when all components come in contact with each other in solution and the solvent is removed. The solvent may be removed either by concentration, precipitation, crystallization, filtration, evaporation, drying, freeze drying, or a combination of the above. The procedure may also include a cooling step, especially if the salt precipitates or crystallizes by itself or when the solution is concentrated. The components may be added to the solution in any order and in stoichiometric amounts or at different ratios. Example of method of preparation is found in Example 1.

Examples of process for forming an organic nutrient salt include, for example, the steps of (a) dissolving homotaurine and at least one other organic, salt-forming, component in a solvent together or successively to obtain a solution, and (b) concentrating the solution. Another example of a process includes the steps of (a) dissolving homotaurine or a salt thereof and a second organic, salt-forming, component in a solvent together or successively to obtain a solution, (b) concentrating and/or cooling the solution until crystals or powder appear, and (c) filtering and drying the crystals or powder obtained. A further example of process includes the steps of (a) dissolving homotaurine or a salt thereof and a second organic, salt-forming component in a solvent together or successively to obtain a solution, (b) adding a second solvent to the solution until crystals appear or a powder precipitates, and (c) filtering and drying the crystals or powder obtained.

For example, choline homotaurinate is prepared by first reacting homotaurine with choline hydroxide in a solvent. The solvent include, without limitation, water and lower alkyl alcohol such as methanol, ethanol and isopropanol, or a mixture of any two or more of them, or a mixture of any one of them with another adequate miscible organic solvent, preferably the solvent is water, methanol, or ethanol. The reaction can be performed at a temperature between about 0° C. and about 100° C., preferably between about 10° C. and about 60° C., more preferably between about 15° C. and about 35° C. or at room temperature.

The crude salt is then obtained by removing the solvent through distillation or evaporation under normal pressure or reduced pressure. Alternatively, the crude product can be precipitated with another solvent or a mixture of other miscible solvents, preferably a solvent in which the product is poorly or not soluble. The crude product may be used as is or may be purified.

Purification can be done using a range of different processes, such as recrystallization, precipitation, chromatographic separation, etc.; preferably recrystallization or precipitation. Examples of solvents suitable for recrystallization or precipitation include, without limitation, lower alkyl alcohol organic solvent, pure or mixed with water, other alcoholic solvent, ketone, ethers, etc. Examples of lower alkyl alcohols for recrystallization include, without limitation, ethanol, propanol, isopropanol, butanol, isobutanol, or mixture of two or more them.

An alternative purification can be done by dissolving the crude product in a solvent (such as water, ethanol, or propanol), followed by precipitation with a second solvent (such as acetone, butanone) or a mixture of other miscible solvents. Another alternative purification involves a direct precipitation of the product by addition of a second solvent or a solvent mixture to the reaction mixture without removing or with partially removing the solvent for the reaction. Further alternative purification is to first precipitate the product from the reaction mixture by adding a second miscible solvent or a miscible solvent mixture, re-dissolving the solid by heating the mixture and then cooled the mixture as done in the normal recrystallization process.

The resulting organic nutrient salt may be administered as is or may be formulated for use in nutrition supplements, as a nutraceutical formulation, or may be used in the preparation of powders or the like, to be used as a food additive.

### III. Compositions Comprising Organic Nutrient Salts

The composition comprising organic nutrient salts may also comprise other ingredients, including, without limitation, excipients, carriers, diluents as well as other health products, such as nutrients (e.g. vitamins, minerals, fatty acids (DHA, EPA, and the like), plant extracts (e.g. a ginko biloba extract, etc)), and the like.

Organic nutrient salts may be used in the preparation of nutraceutical compositions and dietary supplements. Exemplary amounts of homotaurine content in the organic nutrient salt to be administered in one dose include milligram or microgram amounts of homotaurine (in the composition) per kilogram of subject or sample weight (e.g., about 50 micrograms per kilogram to about 500 milligrams per kilogram, about 1 milligram per kilogram to about 100 milligrams per kilogram, about 1 milligram per kilogram to about 50 milligram per kilogram, about 1 milligram per kilogram to about 10 milligrams per kilogram, or about 3 milligrams per kilogram to about 5 milligrams per kilogram). Additional exemplary doses of homotaurine (content in the organic nutrient salt) include doses of about 5 to about 500 mg, or about 25 to about 300 mg, or about 25 to about 200 mg, preferably about 25 to about 150 mg, preferably about 25 to about 100 mg, more preferably about 50, about 100, about 150 mg, about 200 mg or about 250 mg, and, preferably, daily or twice daily, or lower or higher amounts. Exemplary doses for homotaurine (content in the organic nutrient salt) per se include about 2-3 milligram of homotaurine per kilogram of subject (twice daily). Homotaurine doses above refers to the amount of homotaurine content when administering the organic nutrient salt, for example, about 108 mg of carnitine homotaurinate gives a dose of about 50 mg of homotaurine.

a) Nutraceutical Formulations

The organic nutrient salt of the invention is also formulated prior to administration into nutraceutical compositions using techniques and procedures well known in the art. Accordingly, in another embodiment, the present invention relates to compositions comprising effective amounts of an organic nutrient salt as described herein and a suitable vehicle, as well as methods of using and manufacturing such compositions.

The compositions are formulated for oral administration. Suitable acceptable vehicles include, without limitation, any non-immunogenic carrier or diluent suitable for oral administration routes.

Preferably, the material(s) of the invention is orally administered. Formulations of the present invention include those suitable for oral administration. The formulations may conveniently be presented in unit dosage form and may be prepared by any methods well known in the art of pharmacy. Methods of preparing these formulations or compositions include the step of bringing into association a material of the present invention with an acceptable vehicle (e.g. an inert diluent or an assimilable edible carrier) and, optionally, one or more accessory ingredients. In general, the formulations are prepared by uniformly and intimately bringing into association a material of the present invention with finely divided solid carriers and then, if necessary, shaping the product. The amount of the nutritive salt material in such useful compositions is such that a suitable dosage will be obtained.

Formulations of the invention suitable for oral administration may be in the form of capsules (e.g. hard or soft shell gelatin capsule), cachets, pills, tablets, lozenges, powders, granules, pellets, dragees, e.g., coated (e.g., enteric coated) or uncoated, or as pastilles (using an inert base, such as gelatin and glycerin, or sucrose and acacia), each containing a predetermined amount of an organic nutrient salt of the present invention as an active ingredient. An organic nutrient salt of the present invention may also be incorporated directly into the subject's diet. Moreover, in certain embodiments, pellets can be formulated to (a) provide for instant or rapid release of homotaurine and the additional nutrient(s) (i.e., have no coating on them); (b) be coated, e.g., to provide for sustained release over time; or (c) be coated with an enteric coating for better gastrointestinal tolerability.

In solid dosage forms of the invention for oral administration the active ingredient is, for example mixed with one or more nutraceutically acceptable carriers, such as sodium citrate or dicalcium phosphate, or any of the following: fillers or extenders, such as starches, lactose, sucrose, glucose, mannitol, or silicic acid; binders, such as, for example, carboxymethylcellulose, alginates, gelatin, polyvinyl pyrrolidone, sucrose or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the nutraceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. Chewable tablets or else contain one or more components such as sweeteners, flavoring agents and colorants disclosed above.

Prevention of the action of microorganisms can be achieved by various antibacterial and antifungal agents, for example, parabens, chlorobutanol, phenol, ascorbic acid, thimerosal, and the like.

The compositions of this invention can also be administered topically to a subject, e.g., by the direct laying on or spreading of the composition on the epidermal or epithelial tissue of the subject, or transdermally via a “patch”. Such compositions include, for example, lotions, creams, solutions, gels and solids. These topical compositions may comprise an effective amount, usually at least about 0.1%, or even from about 1% to about 5%, of an agent of the invention. Suitable carriers for topical administration typically remain in place on the skin as a continuous film, and resist being removed by perspiration or immersion in water. Generally, the carrier is organic in nature and capable of having dispersed or dissolved therein the nutraceutic agent. The carrier may include nutraceutically acceptable emollients, emulsifiers, thickening agents, solvents and the like.

Other compositions useful for attaining systemic delivery of the subject agents include sublingual and buccal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included. Under ordinary conditions of storage and use, these preparations may contain a preservative to prevent the growth of microorganisms.

Compositions according to the invention may also be coated by conventional methods, typically with pH or time-dependent coatings, such that homotaurine and the additional nutrient(s) are released in the vicinity of the desired location, or at various times to extend the desired action. Such dosage forms typically include, but are not limited to, one or more of cellulose acetate phthalate, polyvinylacetate phthalate, hydroxypropyl methyl cellulose phthalate, ethyl cellulose, waxes, and shellac.

The composition(s) of the invention may be packaged as part of a kit, optionally including a container (e.g. packaging, a box, a vial, etc). The kit may be commercially used according to the methods described herein and may include instructions for use in a method of the invention. Additional kit components may include acids, bases, buffering agents, inorganic salts, solvents, antioxidants, preservatives, or metal chelators. The additional kit components are present as pure compositions, or as aqueous or organic solutions that incorporate one or more additional kit components. Any or all of the kit components optionally further comprise buffers.

b) Use as Food Additive

Organic nutrient salt preparation is added in foodstuffs for increasing the uptake of homotaurine as a neuroprotective and to protect the brain structure associated with memory and learning, to protect memory function, as well as to sustain brain cell health, to maintain verbal skills and comprehension ability and to support planning and execution skills, as well as to provide additional health benefits from the additional nutrient(s) present in the organic nutrient salt. By “foodstuff” is meant any article that can be consumed, for example, eaten, drank, or ingested by a subject.

Examples of foodstuffs which can be enriched with nutrients by adding organic nutrient salt of the invention include, without limitation, beverages (including dry beverage powder), dairy products (e.g., cheese, milk, yogurt, ice cream, etc), bakeries and pastries (e.g, breads, tortillas, pita breads, rolls, cookies, crackers, pies, cakes, nutritional bars, and the like), cereals, noodles, meat products (e.g. sausages, jerked beef, etc), fish products, egg products, nut products, soup mixes, snack foods, salad dressings, sauces, sweets, hard or soft candies, jams or jellies, seasoning (e.g. herbal salt), and the like. Foodstuffs also include food for animals, such as companion pet food. The organic nutrient salt may be added as a powder in the preparation, or may be used as is or formulated to be used as an additive by the consumer.

### IV. Subjects Populations

The term “subject” includes living organisms susceptible to neuronal cell death or neuronal cell loss, memory impairment, loss in verbal skills and comprehension ability, loss in planning and execution skills, etc, living organisms in need of neuroprotection or living organisms in which Aβ-amyloid-related diseases or conditions as defined above can occur. Examples of subjects include humans, chickens, ducks, Peking ducks, geese, monkeys, deer, cows, rabbits, sheep, goats, dogs, cats, mice, rats, and transgenic species thereof. The term “subject” preferably includes animals susceptible to states characterized by neuronal cell death, e.g. mammals, e.g. humans. The animal can be an animal model for a disorder. In preferred embodiments, the subject is a mammal, more preferably a human subject.

The term “human subject” also includes humans susceptible to benefit from homotaurine administration as well as other nutrient(s), including those susceptible to or diagnosed of having an amyloid-β related disease and/or suffering from a neurodegenerative disease, such as Alzheimer's disease, Parkinson's disease, etc. The term human subject equally includes human subjects susceptible to neurodegeneration, neuronal cell loss, or neuronal cell death related or not to amyloid-β deposition, including an aging human subject. The term human subject further equally includes human subjects susceptible to memory impairment, loss in verbal skills and comprehension ability, loss in planning and execution skills, etc.

In certain embodiments of the invention, the human subject is susceptible to benefit from the methods of the invention, and is selected based on this need. A subject in need includes subjects that have been identified as having a disease or disorder related to β-amyloid deposition, has a symptom of such a disease or disorder, or is at risk of such a disease or disorder, and would be expected, based on diagnosis, e.g., medical diagnosis, to benefit from treatment (e.g., curing, healing, preventing, alleviating, relieving, altering, remedying, ameliorating, improving, or affecting the disease or disorder, the symptom of the disease or disorder, or the risk of the disease or disorder).

For example, the human subject may be a human over 30 years old, human over 40 years old, a human over 50 years old, a human over 60 years old, a human over 70 years old, a human over 80 years old, a human over 85 years old, a human over 90 years old, or a human over 95 years old. The subject may be a female human, including a postmenopausal female human, who may be on hormone (estrogen) replacement therapy. The subject may also be a male human. In another embodiment, the subject is under 40 years old.

For example, individuals having or being predisposed to memory or cognitive impairment, age-associated memory impairment or mild cognitive impairment, or forms of dementia, can be identified by the Clinical Dementia Rating (CDR) scale, Mini-mental State Examination (MMSE), Alzheimer's Disease Assessment Scale-Cognitive Subscale (ADAS-Cog), or any other test known in the art, as discussed herein. Baseline scores on suitable metrics including the MMSE and the ADAS together with other metrics designed to evaluate a more normal population can be used to find an at risk population. Another method for identifying an at risk group utilizes an assay for neural thread protein in the urine; see, e.g., Munzar et al., Neurology and Clinical Neurophysiology, Vol. 2002, No. 1. Patients with high risk for Alzheimer's Disease can also be selected from a population by screening for early signs of memory loss or other difficulties associated with pre-Alzheimer's symptomatology, a family history of Alzheimer's Disease, patients with Mild Cognitive Impairment (MCI), genetic risk factors, age, sex, and other features found to predict high-risk for Alzheimer's Disease.

The term “prevention” or “preventing” is also used to describe the administration of a material or composition of the invention to a subject who is at risk of (or susceptible to) such a disease or condition. Subjects amenable to treatment for prevention of the disease or condition include individuals at risk of the disease or condition but not showing symptoms, as well as patients presently showing symptoms. Virtually anyone is at risk of developing a condition related to Amyloid-β, or neurodegeneration if he or she lives long enough. Therefore, the present methods can be administered prophylactically to the general population without any assessment of the risk of the subject patient. The present methods are also useful for individuals who do have a known risk of Alzheimer's disease. Such individuals include those having relatives who have experienced this disease, and those whose risk is determined by analysis of genetic or biochemical markers, including brain plaques diagnosed by imaging methods, e.g., MRI, PET, SPECT etc. Examples of such imaging methods are discussed in Burggren et al., Current Topics in Medicinal Chemistry, vol. 2002, no. 2, pp. 385-393, and Sair et al., Neuroradiology, vol. 46, pp. 93-104 (2002). Alzheimer's disease predisposing factors identified or proposed in the scientific literature include, among others, a genotype predisposing a subject to Alzheimer's disease; environmental factors predisposing a subject to Alzheimer's disease; past history of infection by viral and bacterial agents predisposing a subject to Alzheimer's disease; and vascular factors predisposing a subject to Alzheimer's disease. Genetic markers of risk toward Alzheimer's disease include mutations in the APP gene, particularly mutations at position 717 and positions 670 and 671 referred to as the Hardy and Swedish mutations respectively (see Hardy et al., TINS 20, 154-158 (1997)). Other markers of risk are mutations in the presenilin genes, PS1 and PS2, and ApoE4, family history of AD, hypercholesterolemia or atherosclerosis. The subject may be shown to be at risk by a diagnostic brain imaging technique, for example, one that measures brain activity, plaque deposition, or brain atrophy. The human subject may also be shown to be at risk by a cognitive test such as Clinical Dementia Rating (“CDR”), Alzheimer's disease Assessment Scale-Cognition (“ADAS-Cog”), Disability Assessment for Dementia (“DAD”) or Mini-Mental State Examination (“MMSE”) and/or by any other cognition test known in the art.

In another embodiment, the human subject exhibits no symptoms of Alzheimer's disease. In another embodiment, the subject is at least 40 years of age and exhibits no symptoms of Alzheimer's disease. In another embodiment, the human subject is at least 50 years of age and exhibits no symptoms of Alzheimer's disease.

By using the methods and compositions of the present invention, the levels of amyloid β peptides in a subject's plasma or cerebrospinal fluid (CSF) could be significantly reduced from levels prior to treatment from about 10 to about 100 percent, or even about 50 to about 100 percent, e.g., 15, 25, 40, 60, 70, 75, 80, 90, 95 or 99%. Accordingly, in certain embodiments, the human subject can have an elevated level of amyloid Aβ₄₀ and Aβ₄₂ peptide in the blood and/or CSF prior to a treatment according to the present methods, e.g. Aβ₄₀ levels of greater than about 10 pg/mL, or greater than about 20 pg/mL, or greater than about 35 pg/mL, or even greater than about 40 pg/mL; and Aβ₄₂ levels 30 pg/mL to about 200 pg/mL, or even to about 500 pg/mL. Similarly, according to some embodiments, the methods and compounds of the present invention help reduce the size and/or number of Aβ plaques or Aβ deposits in the brain, from about 10 to about 100 percent, or even about 50 to about 100 percent, e.g., 15, 25, 40, 60, 70, 75, 80, 90, 95 or 99%, when compared to levels prior to treatment. Also, by using the methods of the invention, the healthy cognitive and memory functions may be enhanced, stabilized, improved, or decline may be prevented, as well as general well-being of the individual.

In one embodiment, the composition(s) of the invention is administered at a nutraceutically effective dosage for the prevention or treatment of age-associated memory impairment, mild cognitive impairment, mild-to-moderate cognitive impairment, brain aging, or memory loss. A “nutraceutically effective” dosage stabilizes cognitive and/or memory function or prevents a further decrease in cognitive and/or memory function (i.e., preventing, slowing, or stopping progression).

### VII. Uses of Organic Nutrient Salts and Compositions

Another aspect of the invention pertains to a method for inhibiting neuronal cell death by administering an effective amount of an organic nutrient salt or composition of the present invention. In yet another aspect, the invention pertains to a method for providing neuroprotection to a subject having an Aβ-amyloid related disease or condition, which includes administering or consuming an effective amount of a material or composition of the present invention to the subject, such that neuroprotection is provided. As used herein, the term “neuroprotection” includes protection of neuronal cells of a subject from cell death that may result in initiation of processes such as, but not limited to: the destabilization of the cytoskeleton; DNA fragmentation; the activation of hydrolytic enzymes, such as phospholipase A2; activation of caspases, calcium-activated proteases and/or calcium-activated endonucleases; inflammation mediated by macrophages; calcium influx into a cell; membrane potential changes in a cell; the disruption of cell junctions leading to decreased or absent cell-cell communication; and the activation of expression of genes involved in cell death.

According to a preferred embodiment, the organic nutrient salts and compositions of the present invention are used for one or more of the following: to protect memory function, to protect the brain structure associated with memory and learning, to sustain brain cells health, to maintain verbal skills and comprehension ability, to support planning and execution skills, to treat or prevent an amyloid-β related disease or condition, to regulate production of or levels of amyloid β (Aβ) peptides, and to prevent, reduce, or inhibit amyloid deposition in a subject.

The organic nutrient salts and compositions of the invention may act to ameliorate the course of a disease or condition using any of the following mechanisms (this list is meant to be illustrative and not limiting): slowing the rate of β-amyloid fibril formation or deposition; lessening the degree of β-amyloid deposition; inhibiting, reducing, or preventing amyloid fibril formation; inhibiting neurodegeneration or cellular toxicity induced or not by β-amyloid; inhibiting amyloid induced inflammation in the brain; enhancing the clearance of β-amyloid from the brain; enhancing degradation of Aβ in the brain; or favoring clearance of amyloid protein prior to its organization in fibrils, and decreasing the ratio of Aβ42:Aβ40 in the CSF or plasma. In another embodiment, the invention pertains to a method for improving or preserving cognition and/or memory function in a subject. The method includes administering an effective amount of a salt of the invention, such that the subject's cognition and/or memory function is improved or preserved. The subject's cognition can be tested using methods known in the art such as CDR, MMSE, DAD, and ADAS-Cog or a subscale thereof (e.g. a subset of memory-related tests). Improvement or protection of cognition or memory function is present within the context of the present invention if there is a measurable difference between the performances of subjects using the salts of the invention as compared to members of a placebo group, historical control, or between subsequent tests given to the same subject. The invention also pertains to a method for treating, slowing or stopping a β-amyloid related disease or condition associated with cognitive or memory impairment, by administering to a subject an effective amount of an organic nutrient salt of the invention, wherein the annual deterioration of the subject's cognition as measured by any of the foregoing mentioned test is improved or stabilized.

It is to be understood that wherever values and ranges are provided herein, e.g., in ages of subject populations, dosages, and blood levels, all values and ranges encompassed by these values and ranges, are meant to be encompassed within the scope of the present invention. Moreover, all values in these values and ranges may also be the upper or lower limits of a range.

### VIII. Combination Therapy

In certain embodiments, the organic nutrient salts and compositions according to the invention can be used in concomitantly with at least one therapeutic and/or another nutraceutical agent. The compositions according to the invention and the at least one other therapeutic and/or nutraceutical agent(s) can act additively or, in certain embodiments, synergistically. In certain embodiments, the compositions of the invention can be administered concurrently with the administration of a therapeutic and/or another nutraceutical agent. In certain embodiments, the compositions of the invention can be administered prior or subsequent to administration of a therapeutic and/or another nutraceutical agent. The at least a therapeutic and/or another nutraceutical agent can be effective for treating the same or different disease, disorder, or condition.

Methods of the present invention include administration of one or more organic nutrient salts or compositions of the present invention and one or more therapeutic and/or other nutraceutical agents provided that the combined administration does not inhibit the therapeutic efficacy of the one active ingredient and/or does not produce adverse combination effects.

In certain embodiments, compositions of the present invention can be administered concurrently with the administration of the therapeutic and/or the other nutraceutical therapeutic agent, which can be part of the same composition as, or in a different composition from, that containing the organic nutrient salts of the present invention. In certain embodiments of combination therapy, the combination therapy comprises alternating between administering a composition of the present invention and a composition comprising a therapeutic and/or another nutraceutical agent, e.g., to minimize adverse side effects associated with a particular agent. When an organic nutrient salt or composition of the present invention is administered concurrently with another agent that potentially can produce adverse side effects including, but not limited to, toxicity, the agent can advantageously be administered at a dose that falls below the threshold at which the adverse side effect is elicited.

In certain embodiments, a composition can further comprise substances to enhance, modulate and/or control release, bioavailability, therapeutic efficacy, therapeutic potency, stability, and the like. For example, to enhance nutraceutic effect of homotaurine, the composition can be co-administered with one or more active agents to increase the absorption or diffusion of homotaurine and/or other nutrient(s) from the gastrointestinal tract, or to inhibit degradation thereof in the systemic circulation. In certain embodiments, a composition of the present invention can be co-administered with active agents having a pharmacological effect that enhance the health benefit of homotaurine or another nutrient.

In certain embodiments, organic nutrient salts or compositions of the present invention include, or can be administered to a patient together with, a therapeutic drug that may be available over-the-counter or by prescription. US patent application No. 2005/0031651 (incorporated herein by reference) provide a long but non-exhaustive list of “therapeutic drugs” that can be useful, in combination, according to the invention. Example of therapeutic drugs to be used with the compositions of the present invention are therapeutic drugs useful in the prevention or treatment of Alzheimer's Disease or its symptoms, including but not limited to cholinesterase inhibitors, e.g. donepezil (Aricept™) rivastigmine (Exelon™), Galanthamine (Reminyl™), NMDA receptor antagonists, e.g. memantine (Namenda™), and others, e.g. R-flurbiprofen (Flurizan™). The compositions according to the invention could also be combined with vaccines and antibodies for the prevention or treatment of AD. The composition can also be combined with natural products, nutraceuticals and dietary supplements, including, without limitation, vitamins and minerals, polyunsaturated fatty acids of the Omega group (e.g. omega 3), Galanthamine (also as a nutraceutical), Gotu Kola, dimethylaminoethanol and extracts of Gingko biloba.

### IX. Standard Methods for Testing the Compositions of the Invention.

The organic nutrient salts, and nutraceutical composition or food additive according to the invention can be further analyzed, tested or validated using a variety of in vitro assays, or in vivo assays to confirm their safety, bioavailabity, neuroprotection, their capability to deliver the nutrients, etc. Assays for assessing these parameters are widely described in the literature and they are part of the general knowledge and expertise of the skilled in the art.

For example, biological assays can be conducted to assess whether a composition has a protective effect against neuronal injury or disease. Examples of biological assays include “morphological changes” (e.g. plasma membrane blebbing, cell shape change, loss of substrate adhesion properties, etc), “altered membrane permeability” (e.g. using vital dyes (e.g., propidium iodide and trypan blue), see also, e.g., Haugland, 1996 Handbook of Fluorescent Probes and Research Chemicals, 6th ed., Molecular Probes, OR), “dysfunction of mitochondrial membrane potential” (see, e.g., Haugland, 1996 Handbook of Fluorescent Probes and Research Chemicals, 6th ed., Molecular Probes, OR, pp. 266-274 and 589-594; Kamo et al. (1979) J. Membrane Biol. 49:105; Quinn (1976) The Molecular Biology of Cell Membranes, University Park Press, Baltimore, Md., pp. 200-217; and PCT publication WO 00/19200 to Dykens et al), “caspase activation” (see, e.g., Ellerby et al. (1997) J. Neurosci. 17:6165; Kluck, et al. (1997) Science 275:1132; Nicholson et al. (1995) Nature 376:37; Rosen and Casciola-Rosen (1997) J. Cell Biochem. 64:50; U.S. Pat. No. 5,976,822; Mahajan, et al. (1999) Chem. Biol. 6:401-9; and Xu, et al. (1998) Nucl. Acids. Res. 26:2034-5), “cytochrome C release” (see, e.g., Liu et al. (1996) Cell 86:147), “assays for cell lysis” see, e.g. PCT publication WO 00/70082), “ischemic model systems” (see, e.g., Aarts et al., Science 298:846-850, 2002; Longa, E. Z. et al. (1989) Stroke 20:84; Belayev, L., et al. (1996) Stroke 27:1616; Bederson, J. B. et al. (1986) Stroke 17:472; and De Ryck, M. et al. (1989) Stroke 20:1383), “MTT cytotoxicity assay” (e.g., using the 3-(4,5-dimethylthiazol-2-yl)-2,5-diphenyl tetrazolium bromide (MTT) assay (Trevigen, Gaithersburg, Md.)), “trypan blue cell viability measurement” (see e.g. Yao et al., Brain Res., 889, 181-190 (2001)), and “Determination of Cellular ATP Levels” (e.g. using the ATPLite-M® luminescence assay (Packard BioSciences Co.), and the ATP concentrations are measured on a TopCount NXT® counter (Packard BioSciences Co.)).

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto. The contents of all references, issued patents, and published patent applications cited throughout this application are hereby incorporated by reference. The invention is further illustrated by the following examples, which should not be construed as further limiting.

### EXAMPLES

The Examples set forth herein below provide exemplary preparation of certain representative compositions of the invention.

Unless otherwise indicated, all numbers expressing quantities of ingredients, reaction conditions, concentrations, properties, and so forth used in the specification and claims are to be understood as being modified in all instances by the term “about.” At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contain certain errors resulting from variations in experiments, testing measurements, statistical analyses and such.

The present invention also relates to novel compositions and the preparation thereof. The following detailed examples describe how to prepare the various compositions and/or perform the various processes of the invention and are to be construed as merely illustrative, and not limitations of the preceding disclosure in any way whatsoever. Those skilled in the art will promptly recognize appropriate variations from the procedures both as to solvents, proportions, components, and as to conditions and techniques. In some cases, the components may be commercially available.

Accordingly, the following example is presented to illustrate how organic nutrient salts according to the invention are prepared.

### Example 1

### Preparation of the Organic Nutrient Salt of Carnitine and Homotaurine

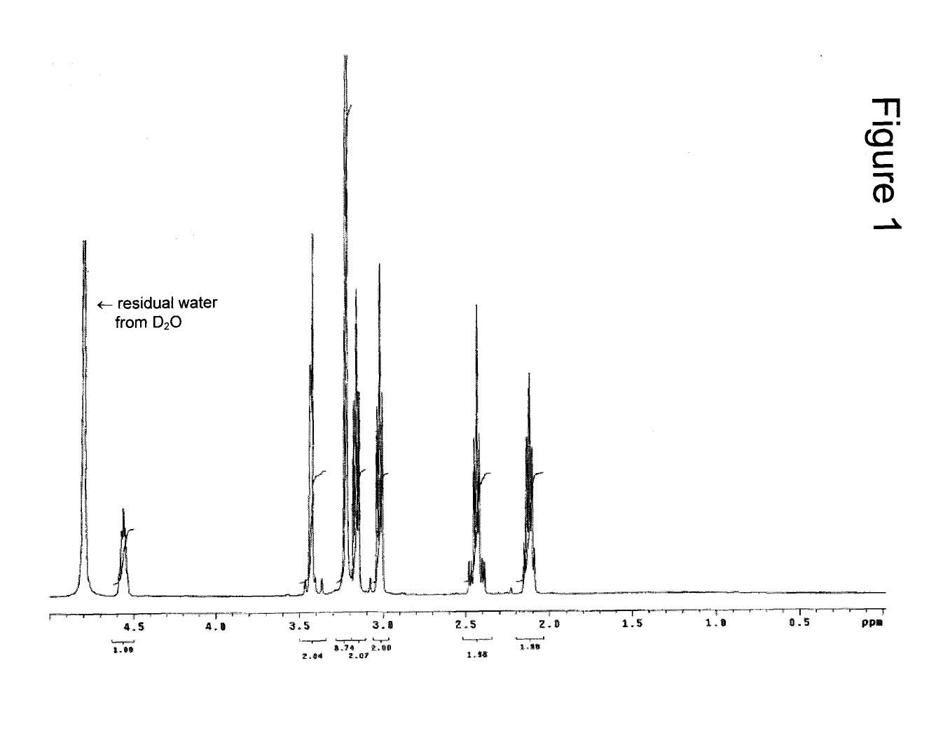

L-carnitine inner salt (10.6 g, 0.066 mole) was dissolved in water (15 mL) with brief heating. To the solution was added homotaurine (9.16, 0.066 mole) with constant stirring. More water (10 mL) was added to give a clear solution. Solvent was evaporated under reduced pressure. The residual solid was suspended in isobutanol (30 mL); and the mixture was concentrated to dryness under reduced pressure. The isobutanol treatment was repeated with a second portion of 15 mL isobutanol. The residual material (18.0 g) was suspended in acetone (200 mL) and the mixture was stirred overnight at room temperature. The solid material was collected by filtration, dried at 60° C. under vacuum, giving a white crystalline powder (17.8 g). ¹H-NMR spectrum of the product was recorded in D₂O (see FIG. 1).

### Example 2

### Preparation (2-hydroxyethyl)trimethylammonium 3-amino-1-propanesulfonate (choline homotaurinate)

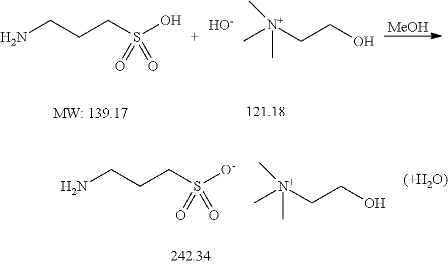

### Procedure 1:

At room temperature, homotaurine (69.6 g, 0.50 moles) was added in one portion to a stirred choline hydroxide solution (143 g of 45 wt. % solution in methanol, 0.53 moles of choline). An additional portion of methanol (50 mL) was added to wash the inner wall of the flask. The mixture was stirred at room temperature for 30 min and a clear solution was obtained (note: in some cases, trace amounts of insoluble materials if any may be removed by filtration before the next step).

The solution obtained was concentrated to dryness under reduced pressure (rotary evaporator) and the solid residual material was further dried at 70° C. (water bath), to give a white solid (between 126 and 129 g).

To the above solid was added isopropanol (300 mL), and the mixture obtained was heated (with effective stirring) to reflux to a clear solution. The solution was allowed to cool to room temperature. The mass of crystals formed was broken into slurry with a metal rod. The slurry was stirred in an ice-water bath for 0.5 to 1 hour. The crystalline solid was collected by filtration, washed with isopropanol (5×25 mL) and dried in a vacuum oven at 60° C. overnight (or 24 to 72 hours), to give a white crystalline powder product, 110.5 to 111 g (91 to 92% yield): mp., 110-111° C.; ¹H NMR (D₂O, reference to DOH at 4.80 ppm) 1.87 (br-pent, 2H), 2.74 (br-t, 2H), 2.95 (br-t, 2H), 3.21 (s, 9H), 3.53 (br-m, 2H), 4.07 (br, 2H); ES-MS (m/z, positive mode) 104, 346; ES-MS (m/z, negative mode) 138, 380.

### Procedure 2:

At room temperature, homotaurine (69.6 g, 0.500 moles; in-house material from Sigma production) was added in one portion to a stirred choline hydroxide solution (141.4 g 45 wt. % in methanol, 0.522 moles of choline hydroxide). Methanol (50 mL) was added to wash the flask's wall. The mixture was stirred for 20 min, with brief-heating, or until the solid disappeared.

The mixture was allowed to cool to room temperature and the solvent was removed on a rotary evaporator (the final bath temperature reached 55° C.). The residual material was co-evaporated with ethanol (100 mL) to give a white solid material (about 133 g).

The solid material was dissolved in hot ethanol (125 mL), giving a clear, colorless solution. The solution was allowed to cool to room temperature, and further cooled in an ice-water bath for 30 min. The solid material was collected through filtration, washed with ice-water cold ethanol (4×25 mL), air-dried for 20 min., and further dried in a vacuum oven at 60° C. overnight, to give a white crystalline powder: 85 g (70%), m.p. 109-110° C.

The filtrate and washings were combined and evaporated to dryness. The residual material was recrystallized from ethanol (40 mL). The solid material was collected, washed ice-water cold ethanol, and dried (vacuum oven), giving the second crop of product (20 g, 16%).

TABLE
Summary of the preparation of choline homotaurinate

| | Solvent for | Co- | Amount of | | Residual^{b} | Yield |
|---|---|---|---|---|---|---|
| Run No. | recrystallization | evaporation | product (g)^{a} | m.p. | solvent (ppm) | (%)^{c} |
| 1 | ethanol | yes | 85.0 | 109-110 | No | 70.2 |
| 2 | isopropanol | yes | 106.7 | n/a | ~1000 | 88.2 |
| 3 | isopropanol | No | 111.0 | 110-111 | ~2400 | 91.7 |
| 4 | isopropanol | No | 110.5 | 110-111 | ~1900 | 91.3 |
| 5 | isopropanol | No | 110.5 | n/a | ~1600 | 91.3 |

^{a}All the reactions were done with a scale of 0.50-mole homotaurine.
^{b}Estimated by proton NMR peak integration. Residual solvent can be reduced or removed entirely through further drying under different conditions.
^{c}Calculated using the amount of product obtained from a single recrystallization; second crop of product not included.

It is understood that the examples and embodiments described herein are for illustrative purposes only and that various modifications or changes in light thereof will be suggested to persons skilled in the art and are to be included within the spirit and purview of this application and scope of the appended claims.

1. An organic nutrient salt of the formula: 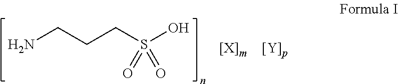
wherein
X is an organic component comprising at least one basic, acidic, cationic or anionic moiety;
Y is an additional component selected from the group consisting of organic or inorganic salt-forming ions, or absent;
m and n are each independently an integer selected from the group consisting of 1, 2, and 3; and
p is an integer selected from the group consisting of 0, 1, 2, and 3, wherein when p is 0, then Y is absent;
or a nutraceutically acceptable salt thereof.

2. The organic nutrient salt of claim 1, having the formula: 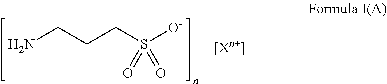
Wherein,
X is an organic component comprising a cationic or protonated basic moiety; and
n is an integer selected from the group consisting of 1, 2, and 3;
or a nutraceutically acceptable salt thereof.

3. The organic nutrient salt of claim 1, wherein X is selected from the group consisting of; L-carnitine, acetyl-L-carnitine, propionyl-L-carnitine, butyryl-L-carnitine, choline, acetylcholine, butyrylcholine, L-arginine and phosphorylcholine.

4. A dietary supplement comprising an organic nutrient salt according to claim 1.

5. A nutraceutical composition comprising an organic nutrient salt according to claim 1 and a nutraceutically acceptable carrier.

6. A preparation comprising an organic nutrient salt according to claim 1 for use in the preparation of food supplements and food stuffs.

7. A method for providing neuroprotection comprising administering to a subject, an organic nutrient salt according to claim 1.

8. A method for protecting memory function comprising administering to a subject, an organic nutrient salt according to claim 1.

9. A method for protecting the brain structure associated with memory and learning comprising administering to a subject an organic nutrient salt according to claim 1.

10. A method for sustaining brain cells health comprising administering to a subject an organic nutrient salt according to claim 1.

11. A method for maintaining verbal skills and comprehension ability comprising administering to a subject an organic nutrient salt according to claim 1.

12. A method for supporting planning and execution skills comprising administering to a subject an organic nutrient salt according to claim 1.

13. A method for treating or preventing a disease or condition in which Amyloid-β proteins or peptides are present comprising administering to a subject an organic nutrient salt according to claim 1.

14. A process of making an organic nutrient salt of claim 1, wherein said process comprises the step of contacting homotaurine or a salt thereof and an organic component having a basic or cationic moiety in solution.

15. The process of claim 14, further comprising isolating the crude product.

16. The process of claim 15, wherein said isolating step is done by evaporation to dryness.

17. The process of claim 15, wherein said isolating step is done by precipitation or crystallization.

18. The process of claim 14, wherein said process further comprises the step of purifying the crude product.

19. The process of claim 18, wherein said purifying step comprises recrystallization.

20. The process of claim 19, wherein said recrystallization comprises the step of dissolving the crude product in a lower alkyl alcohol solvent at a temperature between 35° C. and the boiling point of the lower alkyl alcohol solvent.

21. The process of claim 20, wherein said lower alkyl alcohol is selected from ethanol and isopropanol.

22. The process of claim 21, wherein said lower alkyl alcohol is isopropanol.

23. The process of claim 14, wherein said organic component having a cationic moiety is choline hydroxide.

24. A compound or polymorph thereof, produced by the process of claims 14.
